(19) United States
(12) Patent Application Publication
(10) Pub. No.: US 2026/0137814 A1
(43) Pub. Date: May 21, 2026

(54) FLUORESCENT COMPOUNDS FOR ACCELERATED CANCER THERANOSTIC

(71) Applicant: The Hong Kong University of Science and Technology, Hong Kong (CN)

(72) Inventors: BEN-ZHONG TANG, Hong Kong (CN); HUILIN XIE, Hong Kong (CN); CHEN ZHANG, Hong Kong (CN); TSZ KIN KWOK, Hong Kong (CN)

(21) Appl. No.: 19/121,251

(22) PCT Filed: Sep. 28, 2023
(86) PCT No.: PCT/CN2023/122903

§ 371 Date: Apr. 15, 2025

Related U.S. Application Data
(60) Provisional application No. 63/417,322, filed on Oct. 19, 2022.

(51) Int. Cl. A61K 49/00; A61K 31/433; C09K 11/06

(52) U.S. Cl. CPC A61K 49/0021; A61K 31/433; A61K 49/0093; C09K 11/06; C09K 2211/1081

(57) ABSTRACT

The present invention discloses aggregation-induced emission fluorescent compounds with near-infrared (NIR) emission. This fluorescent compound is then encapsulated by polymer matrix to yield nanoaggregates which is confirmed to show an excellent photothermal conversion efficiency while maintaining a high fluorescence quantum yield. Consequently, the photothermal therapeutic efficacy can be improved by PTA-oriented accumulation of nanoaggregates, compared with the barely enhanced permeability and retention (EPR) effect. The in vitro and in vivo verification further confirmed that the nanoaggregates exhibit superior fluorescence-guided phototheranostics ability to eliminate tumors.

### CROSS REFERENCE TO RELATED APPLICATIONS

This application claims priority to and the benefit of U.S. Provisional Patent Application Ser. No. 63/417,322 filed on Oct. 19, 2022, the disclosure of which is incorporated by reference in its entirety.

### TECHNICAL FIELD

The present invention relates to aggregated-induced emission fluorescent compounds with near-infrared (NIR) emission and applies them into imaging-guided phototheranostic.

### BACKGROUND

Cancer, as one of the most severe health issues in the world, has caused an extremely high mortality rate. Although multifarious interventions, including chemotherapy, radiotherapy, and surgery, have been used to treat cancer, their severe side effects and grim therapeutics outcomes are still far from satisfactory. Therefore, developing more effective cancer therapies is essential but challenging. Fluorescence-guided phototheranostic, which utilizes photoirradiation to achieve cancer diagnostics and therapeutics coinstantaneously, has emerged as a promising tool because of its controllable, non-invasive, and spatio-temporal therapeutic process. However, robust phototherapy systems, which can simultaneously be equipped with diagnostic and therapeutic abilities, are extremely scarce and highly demanded due to the difficulty of tackling the complicated energy decay pathways upon excitation.

Theranostics commonly require multifunctional agents, which is difficult to achieve in single molecules. Utilizing the energy of photoirradiation, theranostic agents can ablate cancer cells under the guidance of single or multi-modal imaging, facilitating cancer diagnosis and treatment simultaneously. Traditionally, several components with individual functions, such as drug and probe, are packaged in one nanoparticle as multifunctional nanomedicine. However, the complexity and cost of this “all-in-one” manner have limited their development for clinical use.

Another critical problem restricting the effective utilization of theranostic agents is their delivery efficiency. Although the enhanced permeability and retention (EPR) effect has been widely acknowledged for decades, only about 0.7% (median) of the administered dose can be delivered to solid tumor. In addition, it requires 24 to 48 hours for EPR to deliver theranostic agents to the targeted tissues, and the targeting modifications are always chemical (click reaction) or biological (antibodies, biomarkers, etc.). Current EPR-based delivery methodologies are no doubt time-consuming, sensitive, complex, expensive, and low effectiveness. Most importantly, it was demonstrated that EPR only exists in small animals such as mice, but there are no EPR in human bodies. A simple and faster delivery method is highly desirable for cancer therapy.

### SUMMARY

The present invention provides simple physical strategy to boost the development of drug development, and reduce the toxic side effect of existing phototheranostics nanomedicine since low dose and milder laser irradiation can be achieved in PTA-enabled accumulation. Thus, the combination of one-for-all NIR-absorbing nanoaggregates and a PTA fast delivery platform can make the most use of the designed materials for disease treatment.

In one embodiment, a fluorescent compound is provided. The fluorescent compound exhibits aggregation-induced emission properties, the fluorescent compound having a backbone structural formula selected from the group consisting of:

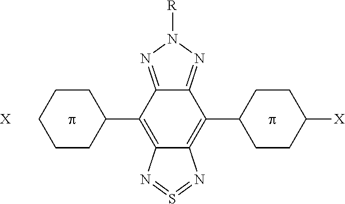

wherein R is selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C(O)—O—, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each 7 is independently selected from the group consisting of:

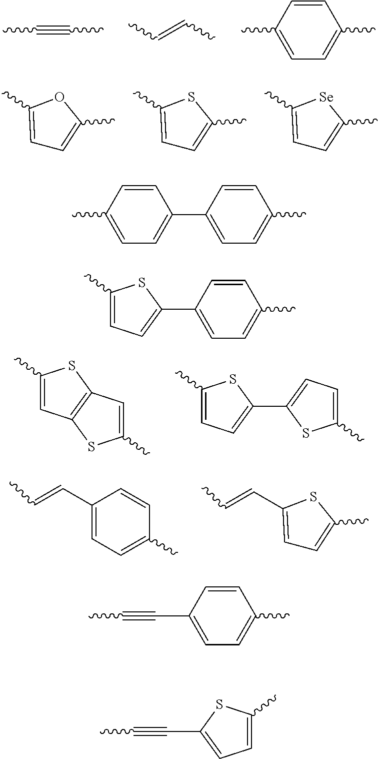

wherein each X is independently selected from the group consisting of:

[chemical structure]

In another embodiment, a theranostic nanoaggregates is provided. The theranostic nanoaggregates comprises:
nanoparticle comprising the the above-mentioned fluorescent compound; and
polymer nanoshell, wherein the polymer nanoshell covers the nanoparticle, the polymer nanoshell is formed by using a polymer matrix to encapsulate the nanoparticle.

In still another embodiment, a method of killing cancer cells is provided. The method comprising:
contacting a target cancer cell with the above-mentioned theranostic nanoaggregates;
imaging the target cancer cell while the theranostic nanoaggregates contacts the target cancer cell using an imaging method selected from the group consisting of fluorescence microscopy, bioluminescence imaging, confocal laser scanning microscopy and photoacoustic microscopy; and
subjecting the target cancer cell to light irradiation while the theranostic nanoaggregates is contacting the target cancer cell converts light to heat to kill the target cancer cell.

In further another embodiment, a method of stopping tumor growth or inhibiting tumor growth or eliminating tumor in a mammal is provided. The method comprising:
administering the above-mentioned theranostic nanoaggregates to the mammal;
contacting a tumor site with the theranostic nanoaggregates;
locating the tumor site using an imaging method after the tumor site is contacted with the theranostic nanoaggregates;
subjecting the tumor site to an initial pulsed light irradiation to enhance the vascular permeability near the tumor site, so as to drive enough theranostic nanoaggregates to accumulate on the tumor site; and
subjecting the tumor site to a subsequent continuous light irradiation while enough theranostic nanoaggregates at the tumor site converts light to heat to rise the temperature of the tumor site to a desired temperature, so as to stop or inhibit the growth of the tumor, or to eliminate the tumor.

The above description is only an outline of the technical schemes of the present invention. Preferred embodiments of the present invention are provided below in conjunction with the attached drawings to enable one with ordinary skill in the art to better understand said and other objectives, features and advantages of the present invention and to make the present invention accordingly.

### BRIEF DESCRIPTION OF THE DRAWINGS

The present invention can be more fully understood by reading the following detailed description of the preferred embodiments, with reference made to the accompanying drawings, wherein:

FIG. 1 Synthetic route of TBT-2(1P-DPA), TBT-2(2P-DPA), and TBT-2(TP-DPA).

FIG. 2 Chemical structure of TBT-2(TP-DPA).

FIG. 3a Molecular design principle and chemical structures of TBT-2(1P-DPA), TBT-2(2P-DPA), and TBT-2(TP-DPA). FIG. 3b Optimized S₀ geometries and illustration of the frontier molecular orbitals (LUMOs and HOMOs) determined by the B3LYP/6-31G* level of theory. FIG. 3c Normalized absorption spectra and FIG. 3d normalized PL spectra of the compounds in THF solution (10 μM). FIG. 3e Plots of absorption maximum (hollow circle) and emission maximum (solid circle) in THF and the corresponding Stokes shift of the three compounds.

FIG. 4a The schematic illustration of the preparation of PTA nanoaggregates. FIG. 4b DLS distribution of the PTA nanoaggregates in water. Inset: TEM image of the nanoaggregates. FIG. 4c Normalized absorption and PL spectra of the PTA nanoaggregates. FIG. 4d Photothermal conversion curve of the PTA nanoaggregates. FIG. 4e The photoacoustic spectrum of the PTA nanoaggregates in an aqueous solution. Insets: photoacoustic images of PTA nanoaggregates at concentrations of 0, 50, 100, 200, and 500 μg mL⁻¹.

FIG. 5 The calculation of photothermal conversion efficiency of TBT-2(TP-DPA) nanoaggregates. The PTA nanoaggregates (1 mg mL⁻¹) was upon 808 nm irradiation (1 W cm⁻²) for 5 minutes, and then the laser was shut off. Time constant for heat transfer of PTA nanoaggregates was calculated to be τ_{S}=186.1 s.

FIG. 6 The photoacoustic spectrum of TBT-2(TP-DPA) nanoaggregates in aqueous solution.

FIG. 7a The dual-wavelength (532 nm and 840 nm) ORPAM imaging of the tumor from a representative mouse intravenously injected with TBT-2(TP-DPA) PTA nanoaggregates (1 mg mL⁻¹, 200 μL), followed by a series of sequential scanning, scale bars: 1 mm. FIG. 7b Quantitative analyses of the accumulation of the nanoaggregates in the flank tumors by the photoacoustic intensity in panel a.

FIG. 7c The NIR-II fluorescence imaging of tumor in EPR group post i.v. of PTA nanoaggregates (1 mg mL⁻¹, 200 μL). FIG. 7d Quantitative analyses of the time-dependent nanoaggregates accumulation in the flank tumors in the EPR group by fluorescence intensity in panel c. FIG. 7e The NIR-II fluorescence imaging of bilateral tumors post intravenous injection (i.v.) of the prepared nanoaggregates (1 mg mL⁻¹, 200 μL). The right tumor was treated with an 808 nm pulse laser (ORPAM and ORPAM+PAT group) or CW laser, while no treatment was performed on the left tumor. FIG. 7f Quantitative analyses of the accumulation of the nanoaggregates in the flank tumors by fluorescence intensity in panel c and e.

In vitro photothermal performance of the PTA nanoaggregates. FIG. 8a The temperature increases of PTA nanoaggregates with the different power densities (from lower to upper: 0.2, 0.4, 0.6, 0.8 and 1 W cm⁻²) under 808 nm laser irradiation for 5 min. FIG. 8b The temperature increases of the nanoaggregates with different concentrations (from lower to upper: 0, 0.03125, 0.0625, 0.125, 0.25, 0.1 and 1 mg mL⁻¹) under 808 nm laser irradiation (1 W cm⁻²) for 5 min. FIG. 8c Photothermal conversion curves in five NIR laser on/off cycles (808 nm). FIG. 8d Flow cytometry analysis of 4T1 cells after various treatments, including cells without treatment (control), PTA nanoaggregates-treated cells (PTA nanoaggregates), 808 nm CW laser-treated cells (808 nm CW laser), and cells treated with PTA nanoaggregates and 808 nm CW laser (PTA nanoaggregates+808 nm CW laser. FIG. 8e Live/dead assay results of 4T1 cells after varied treatments as in d. Laser power: 1 W cm⁻². Scale bars: 50 μm.

FIG. 9 Photothermal performance of PTA nanoaggregates in vitro. IR thermal images of TBT-2(TP-DPA) nanoaggregates with different concentrations (0.03125-1 mg mL⁻¹) under an 808 nm laser irradiation (1 W cm⁻²) for 5 min.

FIG. 10. The viabilities of 4T1 cells after treatment with TBT-2(TP-DPA) nanoaggregates at concentrations of 0, 10, 25, 50, 100 and 250 μg mL⁻¹ for 24 h.

PTT efficacy of PTA nanoaggregates against subcutaneous 4T1 tumors in vivo. FIG. 11a IR thermal images of 4T1 tumor-bearing mice under an 808 nm (1 W cm⁻²) laser irradiation for 10 min after various treatments. FIG. 11b The corresponding temperature change curves at the tumor site of 4T1 tumor-bearing mice in each group. FIG. 11c Tumor growth and FIG. 11d body weight curves of different groups of mice. FIG. 11e Representative digital images of mice in each experimental group at different time points and corresponding H&E staining of tumor sections. Data represent as mean±s.d. (n=3-5 mice for all groups); 0.01<*P<0.05, 0.001<**P<0.01, ***P<0.001. Scale bars: 50 μm.

FIG. 12 The digital images of the tumor on the 14th day with various treatments in photothermal therapy. Note: (a) control, (b) 840 nm pulse laser+808 nm CW laser, (c) PTA, (d) EPR 24 h+PTT and (e) PTA+PTT.

FIG. 13 The in vivo biosafety analysis of PTA nanoaggregates. Blood routine analysis data, containing the numbers of WBC, RBC, PLT, Lym and HGB, MCH (mean corpsular hemoglobin), MCV (mean corpsular volume), MCHC (mean corpsular hemoglobin concentration) of control and PTA nanoaggregates treated mice.

FIG. 14 The in vivo biosafety analysis of PTA nanoaggregates. Serum biochemistry data, containing ALT, AST, total protein, ALB, creatinine (CREA), total cholesterol, blood urea nitrogen (BUN) and triglycerides levels of control and PTA nanoaggregates treated mice were measured.

FIG. 15 H&E-staining images of major organs (heart, liver, spleen, lung and kidney) of the mice collected from the control group and the PTA nanoaggregates-treated mice at 7 day and 14 day after intravenous injection of PTA nanoaggregates. Scale bars: 100 μm.

### DETAILED DESCRIPTION

### Definitions

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

The term “λₑₓ” as used herein refers to excitation wavelength.

The phrase “aggregation caused quenching” or “ACQ” as used herein refers to the phenomenon wherein the aggregation of π-conjugated fluorophores significantly decreases the fluorescence intensity of the fluorophores. The aggregate formation is said to “quench” light emission of the fluorophores.

The phrase “aggregation induced emission” or “AIE” as used herein refers to the phenomenon manifested by compounds exhibiting significant enhancement of light-emission upon aggregation in the amorphous or crystalline (solid) states whereas they exhibit weak or almost no emission in dilute solutions.

“Emission intensity” as used herein refers to the magnitude of fluorescence/phosphorescence normally obtained from a fluorescence spectrometer or fluorescence microscopy measurement; “fluorophore” or “fluorogen” as used herein refers to a molecule which exhibits fluorescence; “luminogen” or “luminophore” as used herein refers to a molecule which exhibits luminescence; and “AlEgen” as used herein refers to a molecule exhibiting AIE characteristics.

As used herein, a “donor” material refers to an organic material, for example, an organic nanoparticle material, having holes as the majority current or charge carriers.

As used herein, an “acceptor” material refers to an organic material, for example, an organic nanoparticle material, having electrons as the majority current or charge carriers.

As used herein, a “theranostic agent” refers to an organic or inorganic material, for example, an nanoparticle or nanoaggregate material, having both diagnostic and therapeutic capabilities.

Unless defined otherwise, all technical and scientific terms used herein have the same meaning as commonly understood to one of ordinary skill in the art to which the presently described subject matter pertains.

### Fluorescent Compounds and Fluorescent Nanoaggregates

Nanoaggregates, including the cluster of molecules and nanoparticles, have aroused much research interest due to the distinct properties from materials at the molecular level. For example, the nanocrystals of drugs can address the issues of insolubility and bioavailability, which have better effects than molecules in clinics. The nanoaggregates of molecules with aggregation-induced emission (AIE) characteristics can emit luminescence with higher intensity compared to their dispersed molecules, which has advanced the field in imaging, diagnostics and therapies, and optoelectronic devices during the past 20 years.

However, aggregate science is still unexplored since aggregates receive much less attention even though they may be the smallest entities that are genuinely working, especially in multifunctional material applications. Aggregates can either be homo- or heterogeneous. Homo-aggregates of a single component can exhibit properties that molecules do not possess by packing them together. Most AIE materials are homo-aggregates, where different parts of the nanoaggregates can perform various duties. The findings of J-aggregates also indicate that packing mode can affect the behaviors of aggregates. Hetero-aggregates can offer more possibilities because of the interaction between components, while the outcome is hard to anticipate due to the system complexity. Hence, controlling multifunctionality without altering the original chemical formulation and synthesis of the nanoaggregates is worth considering.

Homo-aggregates derived from a single component are thus highly demanded, in which their properties and functionalities are controllable and predictable. Aggregation-induced emission luminogens (AlEgens) are ideal design templates for the “one-for-all” construction of phototheranostic agents, which perfectly address the challenge of aggregation-caused quenching (ACQ) and exhibit low in vivo side toxicities. AlEgens are often non-emissive in solution due to the consumption of the excited state energy via non-radiative relaxation by intramolecular motion. This uncommon feature makes AlEgens ideal for fabrication of fluorescent nanoaggregates with ultrahigh brightness and photobleaching threshold.

The present subject matter contemplates a fluorescent compound having aggregation-induced emission (AIE) characteristics and exhibiting near-infrared emission. Near-infrared (NIR) fluorescence imaging exhibits lower tissue autofluorescence interference and deeper tissue penetration depth than visible light, so it has drawn extensive attention. The compound can be in nanoparticle form. The fluorescent compound can be in nanoparticle form.

Also provided are fluorescent nanoaggregates comprising:
nanoparticle comprising a fluorescent compound; and
polymer nanoshell, wherein the polymer nanoshell covers the nanoparticle, the polymer nanoshell is formed by using a polymer matrix to encapsulate the nanoparticle. The fluorescent nanoaggregates are also referred to herein as “theranostic nanoaggregates”.

Encapsulating the fluorescent compound in a polymer matrix can enhance the intra-particle microenvironment and thereby provide enhanced fluorescence.

In an embodiment, the fluorescent compounds have a backbone structural formula selected from the group consisting of:

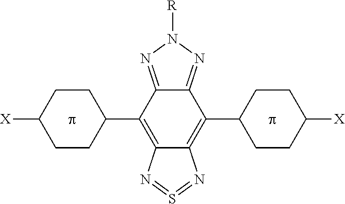

wherein R is selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C(O)—O—, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each 71 is independently selected from the group consisting of:

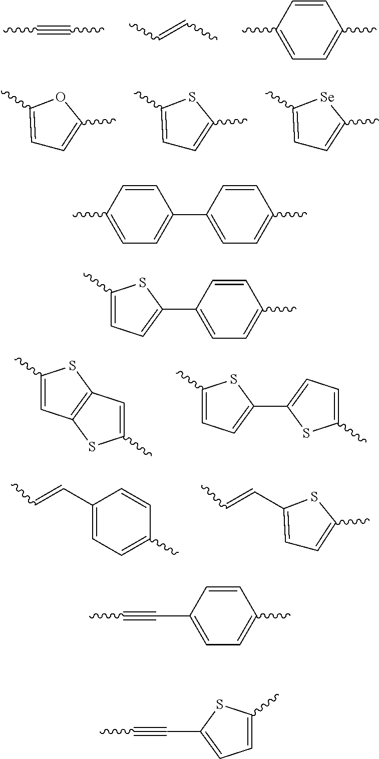

wherein each X is independently selected from the group consisting of:

[chemical structure]

In a further embodiment, the fluorescent compound comprises:

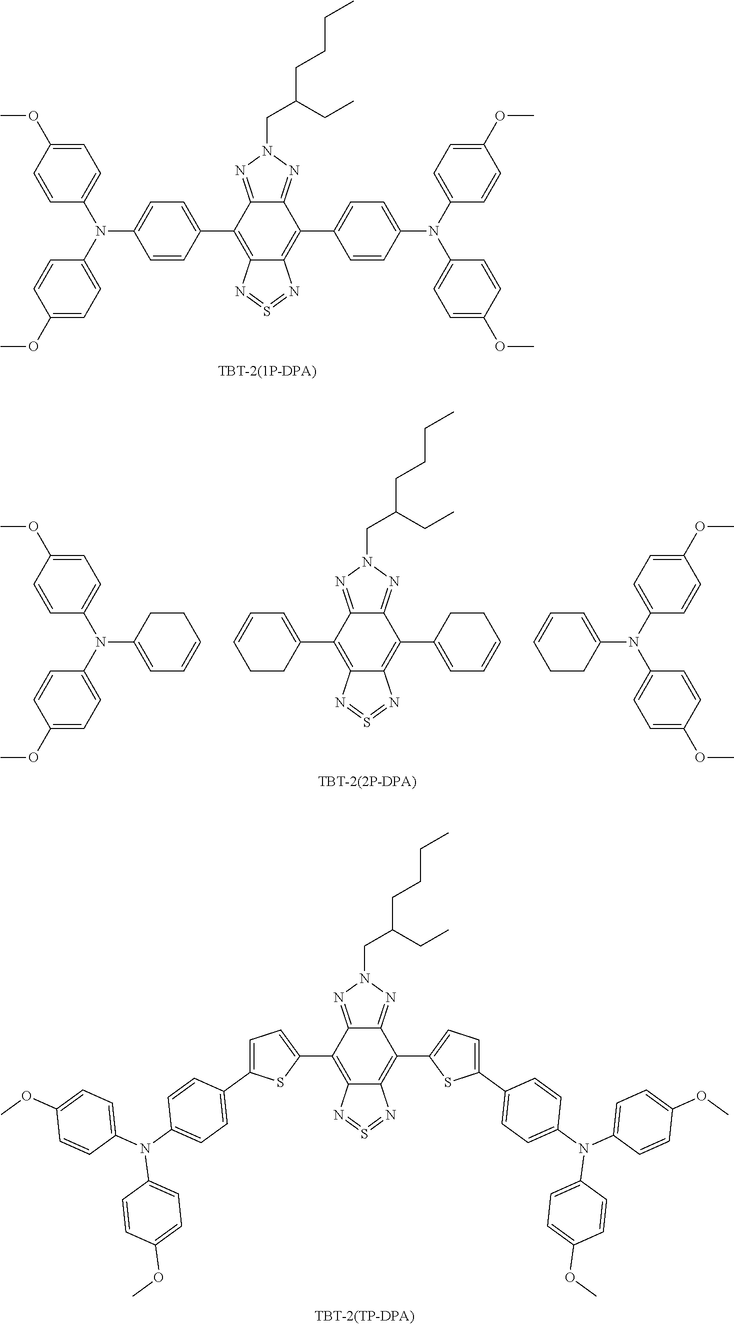

In yet another embodiment, the present subject matter relates to a theranostic nanoaggregates comprising:
nanoparticle comprising a fluorescent compound; and
polymer nanoshell, wherein the polymer nanoshell covers the nanoparticle, the polymer nanoshell is formed by using a polymer matrix to encapsulate the nanoparticle. The fluorescent compound having a backbone structural formula selected from the group consisting of:

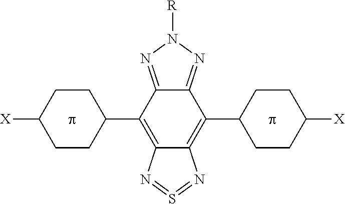

wherein R is selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C(O)—O—, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each 71 is independently selected from the group consisting of:

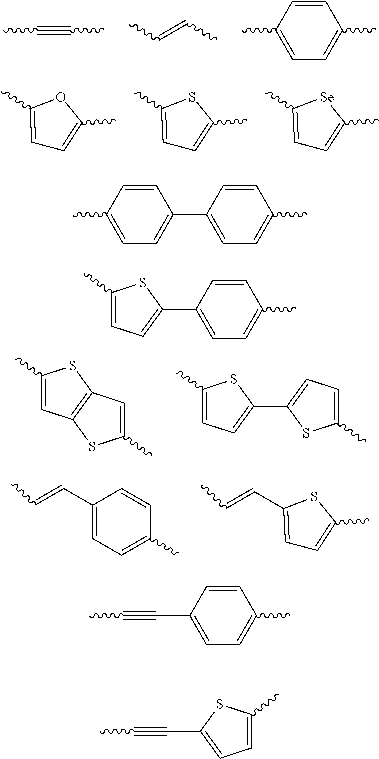

wherein each X is independently selected from the group consisting of:

[chemical structure]

In a further embodiment, the fluorescent compound in the theranostic nanoaggregates is selected from the group consisting of:

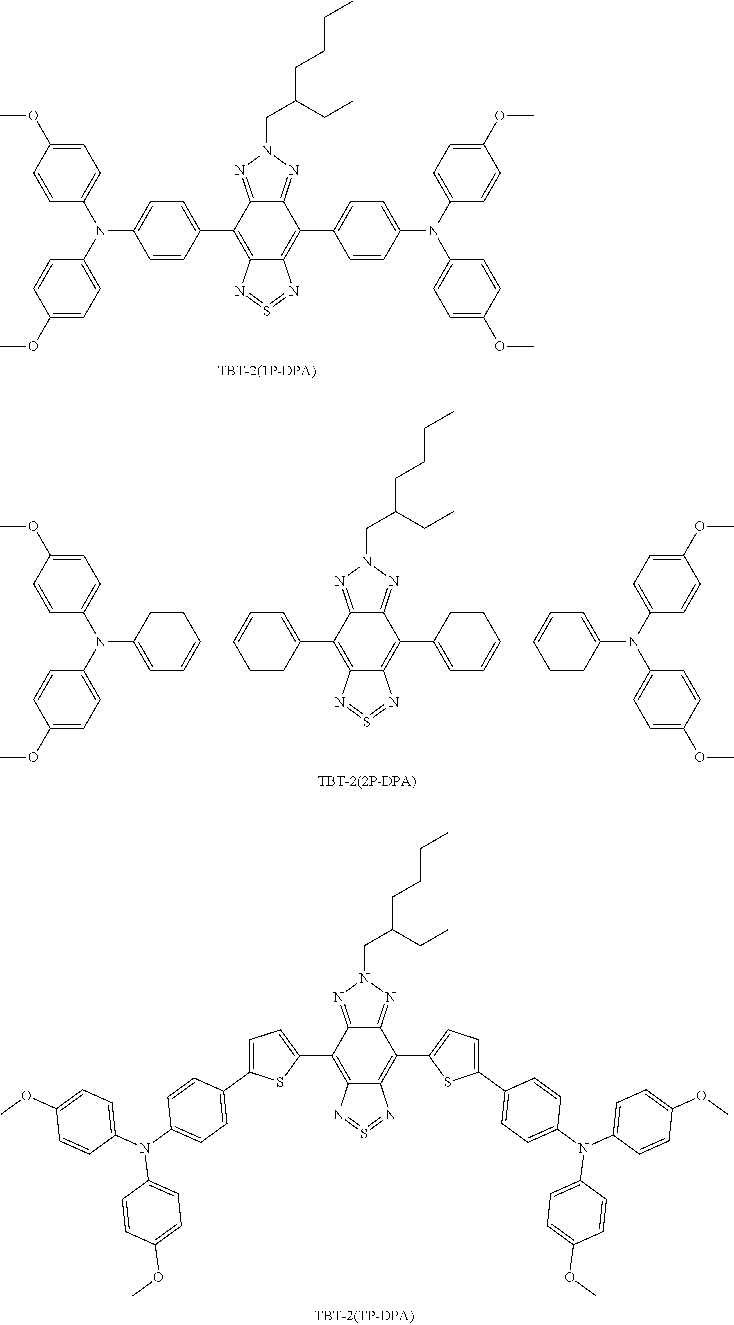

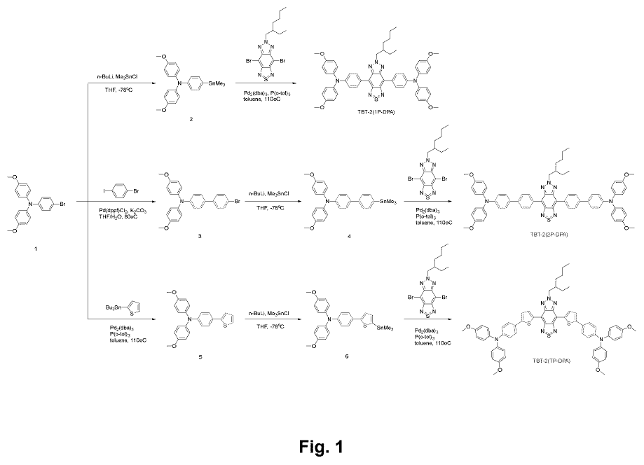

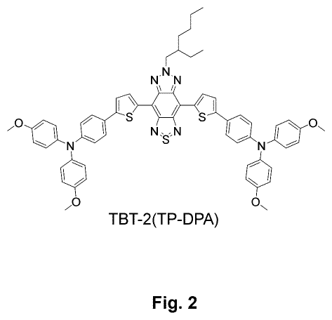

An exemplary reaction scheme for preparing TBT-2(1P-DPA), TBT-2(2P-DPA) and TBT-2(TP-DPA) compounds is illustrated in FIG. 1.

In a photoexcitation process, one typical radiative pathway for the molecule to deal with the energy received after excitation is fluorescence (FL). Another pathway is non-radiative dissipation to afford the photothermal effect, which could be used as photothermal therapy (PTT).

In a preferred embodiment of this invention, we provided a one-for-all AIE system for imaging-guided phototheranostic with excellent photothermal conversion efficiency while maintaining appropriate fluorescence quantum yield. By developing a series of D-(π)-A-(π)-D organic small molecules with well-designed π bridges, named TBT-2(1P-DPA), TBT-2(2P-DPA), and TBT-2(TP-DPA), as the candidates for one-for-all phototheranostic agents. It turns out that TBT-2(TP-DPA) with the thiophene-containing bridge exhibits the most balanced NIR imaging and therapeutic potential. The calculation results suggest that TBT-2(TP-DPA) shows the strongest D-A interaction and the favorable intramolecular charge transfer effect, which benefits the longer emission wavelength extended to ˜1500 nm. After simply encapsulation with DSPE-PEG₂₀₀₀, the nanoaggregates of TBT-2(TP-DPA) can demonstrate photothermal conversion efficiency of up to 51% while keeping an acceptable photoluminescence quantum yield in the NIR region as well.

The fluorescent compound TBT-2(TP-DPA) exhibits NIR-I excitation and NIR-II emission. The remarkable headway made in the second near-infrared region (NIR-II, 1000-1600 nm) has promoted the development of biomedical imaging significantly. NIR-II fluorescence imaging possesses a number of merits which prevail over the traditional and NIR-I (650-950 nm) imaging modalities in fundamental research, such as reduced photon scattering, as well as auto-fluorescence and improved penetration depth.

Cancer Diagnostics and/or Cancer Therapy

The theranostic nanoaggregates described herein can be beneficial in cancer diagnostic and phototheranostic applications, particularly with respect to NIR imaging-guided cancer surgery with excellent photothermal conversion efficiency while maintaining appropriate fluorescence quantum yield. Image-guided cancer surgery using NIR fluorescence has been verified to be feasible during clinical cancer surgery, and holds great promise for successful outcomes in cancer surgery. The theranostic nanoaggregates described herein can be used as efficient NIR fluorescent probes that meet the necessary requirements of image-guided cancer surgery.

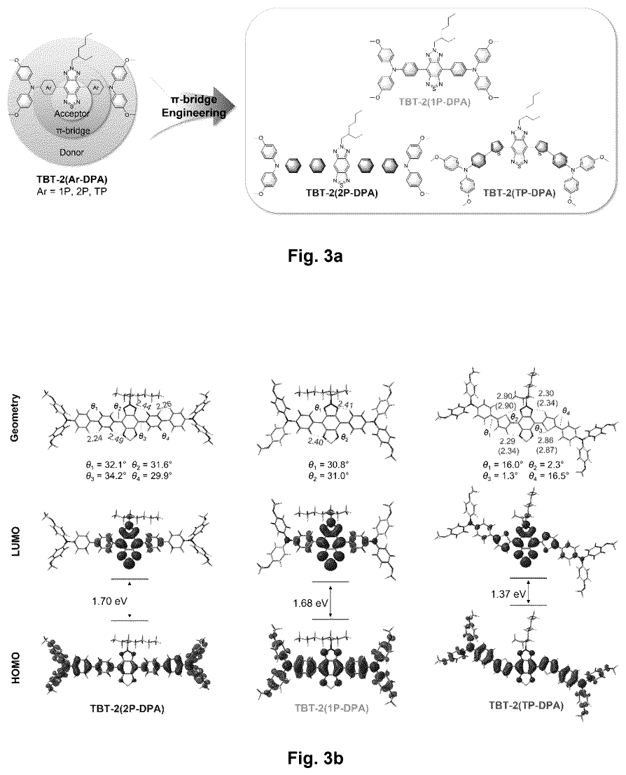

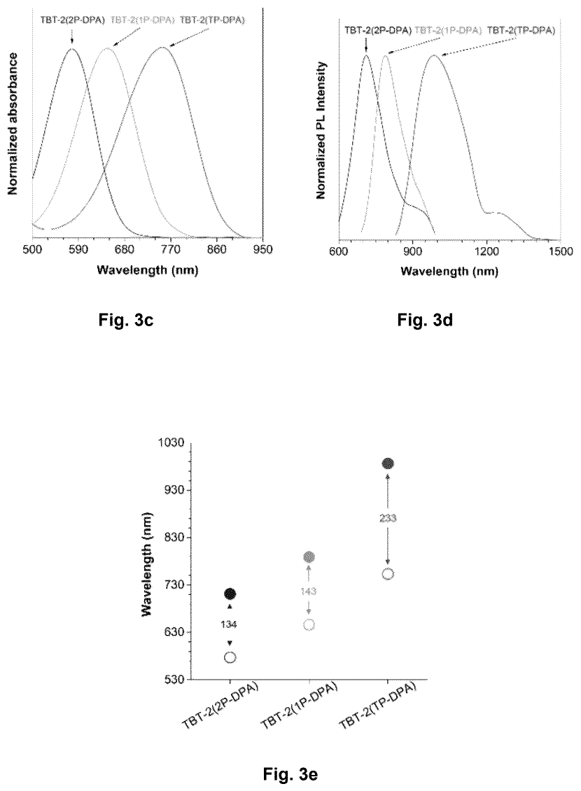

As described herein, we measure the absorption and emission spectrum of TBT-2(1P-DPA), TBT-2(2P-DPA), and TBT-2(TP-DPA) in various polar solvents. It turns out that TBT-2(1P-DPA) and TBT-2(2P-DPA) possess absorption peaks at 580 and 660 nm in THE solution, respectively, and their absorption both ended up to 800 nm (FIG. 3c). However, the absorption peak of TBT-2(TP-DPA) was around 800 nm, and the whole absorption wavelength can be extended to NIR-1 region (FIG. 3c). Expectedly, the emission spectrum of TBT-2(1P-DPA), and TBT-2(2P-DPA) both ends below 1000 nm, while the emission maximum of TBT-2(TP-DPA) was 1046 nm when upon 808 nm excitation (FIG. 3d). Therefore, TBT-2(TP-DPA) is an ideal candidates as the template of NIR-II theranostic agents.

Under 808 nm excitation, the absolute quantum yield of TBT-2(TP-DPA) in the NIR region was determined to be 10.4% in toluene. The quantum yield (QY) value of the molecule in other solvents and its solid all indicated its superior fluorescence emission capability in the NIR region, making it the preferred probe for in vivo imaging.

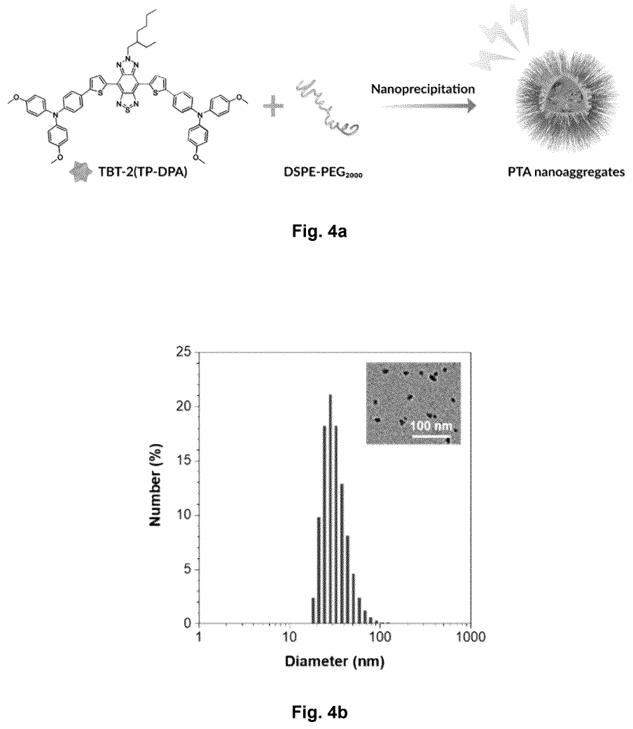

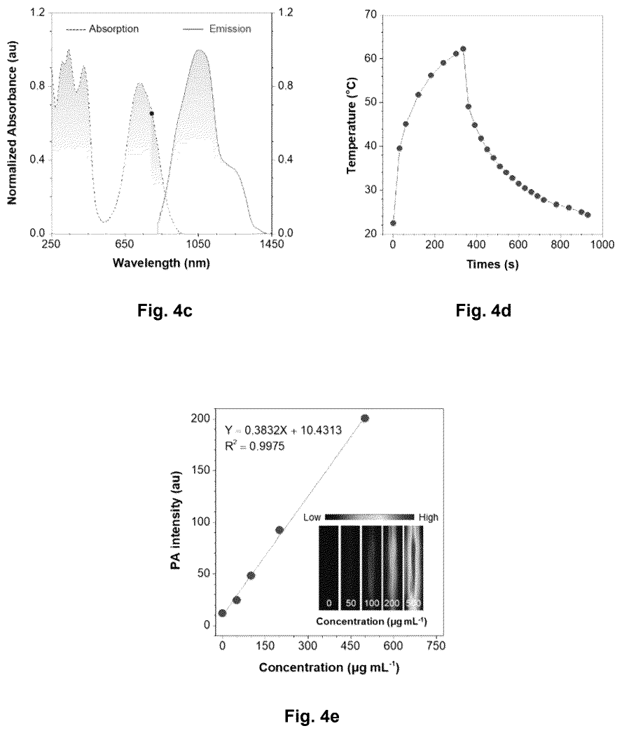

TBT-2(TP-DPA) theranostic nanoaggregates is prepared by simply encapsulating TBT-2(TP-DPA) with DSPE-PEG₂₀₀₀ (FIG. 4a). The nanoaggregates of TBT-2(TP-DPA) were prepared with an average size of around 32 nm and evenly morphology shown by dynamic laser scanning (DLS) and transmission electron microscopy (TEM) (FIG. 4b). The absorption and emission spectrum of the nanoaggregates were recorded (FIG. 4c), which is similar with that of pure organic molecules.

According to an embodiment, the present subject matter relates to a method of killing cancer cells, which can include contacting the theranostic nanoaggregates with a target cancer cell, imaging the target cancer cell while the theranostic nanoaggregates contacts the target cancer cell, and subjecting the target cancer cell to light irradiation while the theranostic nanoaggregates contacts the target cancer cell to kill the target cancer cell. The imaging method can be selected from fluorescence microscopy, bioluminescence imaging, confocal laser scanning microscopy and photoacoustic microscopy. The theranostic nanoaggregates can be combined with a buffer solution prior to contacting the target cancer cell. The target cancer cell can be in live mammals.

The light irradiation can be near-infrared light irradiation or laser irradiation.

Current EPR-based delivery methodologies are no doubt time-consuming, sensitive, complex, expensive, and low effectiveness. Most importantly, it was demonstrated that EPR only exists in small animals such as mice, but there are no EPR in human bodies. A simple physical delivery method is highly desired for clinical use.

Unexpectedly, based on the photoinduced thermoacoustic processes, it was found that the photoacoustic effect can not only be used as an imaging technique, but also be a driving force for fast delivery in vitro and in vivo. The mechanisms may lie in two aspects. Frist, the vasculature is not a seamless structure, their permeability can be enhanced or even damaged by the heat from the photothermal process. There are already some previous studies discussing photothermal agents for enhancing vascular permeability, however the potential risks must be considered because overheating from continuous-wave lasers can directly damage the blood vessels and do harm to the biological tissues.

However, if we use a pulse laser, the photothermal effect is negligible because the photoinduced stimulation is just at the magnitude nanosecond, and the interval between two pulses is of microsecond, leaving enough time for the transient heat to be dissipated into the surrounding media. As a result, the photoinduced thermoacoustic processes will generate a PTA-field or PTA-force (or as researchers in the photoacoustic field may call it photoacoustic radiation force, PAF), to enhance the vascular permeability in a non-invasive, specific, and transient manner. It can trigger sufficient agents to aggregate in the tumor tissue with high efficiency (within one hour), which solves the problems of time consumption and low effectiveness of traditional EPR.

According to an embodiment, the present subject matter relates to a method of stopping tumor growth or inhibiting tumor growth or eliminating tumor in a mammal, which can include administering the theranostic nanoaggregates to the mammal; contacting a tumor site with the theranostic nanoaggregates; locating the tumor site using an imaging method after the tumor site is contacted with the theranostic nanoaggregates; subjecting the tumor site to an initial pulsed light irradiation to enhance the vascular permeability near the tumor site, so as to drive enough theranostic nanoaggregates to accumulate on the tumor site, and subjecting the tumor site to a subsequent continuous light irradiation while enough theranostic nanoaggregates at the tumor site converts light to heat to rise the temperature of the tumor site to a desired temperature, so as to stop or inhibit the growth of the tumor, or to eliminate the tumor. The desired temperature is preferred higher than 55 degrees Celsius. The theranostic agent can be administered by intravenous injection. The imaging method comprises at least one of fluorescence microscopy, bioluminescence imaging, confocal laser scanning microscopy and photoacoustic microscopy.

The light irradiation can be near-infrared irradiation or laser irradiation.

The initial pulsed light irradiation comprises a sequence of light pulses more than 3. The peak irradiation intensity is higher than 0.5 W/cm² and lower than 100 W/cm². The effective pulse length is between 5 microseconds to 5 seconds.

The off time between each two adjacent light pulses sufficient to allow the transient heat of the vasculature near the tumor site to be dissipated into the surrounding media, so as to avoid overheating and damaging the vasculature near the tumor site.

The combined duration of the initial pulsed light irradiation and the subsequent continuous light irradiation is less than one hour.

It is worth mentioning that in nonradiative decay, the acoustic wave from heat generation can not only assist photoacoustic imaging (PAI), but also be a guiding force for particle delivery in vitro and in vivo. Moreover, it has been validated that many kinds of nanoparticles or nanoaggregates with the photoacoustic response, no matter ICG, small molecules, polymers or AuNPs, can be triggered by this photoacoustic radiation force.

According to an embodiment, the present subject matter relates to a method of stopping tumor growth or inhibiting tumor growth or eliminating tumor in a mammal, which can include administering theranostic agents to the mammal; contacting a tumor site with the theranostic agents; locating the tumor site using an imaging method after the tumor site is contacted with the theranostic agents; subjecting the tumor site to an initial pulsed light irradiation to enhance the vascular permeability near the tumor site, so as to drive enough theranostic agents to accumulate on the tumor site, and subjecting the tumor site to a subsequent continuous light irradiation while enough theranostic agents at the tumor site converts light to heat to rise the temperature of the tumor site to a desired temperature, so as to stop or inhibit the growth of the tumor, or to eliminate the tumor. The desired temperature is preferred higher than 55 degrees Celsius. The theranostic agents can be administered by intravenous injection. The imaging method comprises at least one of fluorescence microscopy, bioluminescence imaging, confocal laser scanning microscopy and photoacoustic microscopy.

The theranostic agents comprises nanoparticles or nanoaggregates with the photoacoustic response. The theranostic agents can comprise ICG, small molecules, polymers, AuNPs or AlEgens.

The light irradiation can be near-infrared irradiation or laser irradiation.

The initial pulsed light irradiation comprises a sequence of light pulses more than 3. The peak irradiation intensity is higher than 0.5 W/cm² and lower than 100 W/cm². The effective pulse length is between 5 microseconds to 5 seconds.

The off time between each two adjacent light pulses sufficient to allow the transient heat of the vasculature near the tumor site to be dissipated into the surrounding media, so as to avoid overheating and damaging the vasculature near the tumor site.

The following examples are provided to illustrate further and to facilitate the understanding of the present teachings and are not in any way intended to limit the invention.

### EXAMPLES

### Example 1—Synthesis and Characterization

The Synthetic route of TBT-2(1P-DPA), TBT-2(2P-DPA), and TBT-2(TP-DPA) are shown in FIG. 1.

To construct D-π-A-π-D NIR-II-emissive molecules, the benzobisthiadiazole (BBT) unit is the most used acceptor moiety. Although it exhibits strong electron-withdrawing ability and quinoidal character that are beneficial for red-shifted absorption and emission, it usually results in low fluorescence quantum yield. Thus, developing alternative NIR-II acceptor cores with bright NIR-II fluorescence is of vital significance. Here, we applied another strong electron-withdrawing unit named thiadiazolobenzotriazole (TBT) as the acceptor core to design a series of NIR-emissive molecules. Starting from diphenylamine (DPA) as the electron-donating unit, we employed three different aromatic moieties, namely phenyl, biphenyl, and phenylthienyl groups, as the Tr-units to bridge the TBT acceptor and the DPA donor. Despite the simple structures of these r-units, it is anticipated that they endow the molecules with various electronic effects and aggregation behavior when incorporated into the D-Tr-A-Tr-D molecules. The three final products, TBT-2(1P-DPA), TBT-2(2P-DPA), and TBT-2(TP-DPA), can be synthesized via facial Stille coupling reactions with high yield and easy purification, as shown in FIG. 3a.

Density-functional theory (DFT) calculations with B3LYP functional and 6-31 G* basis sets to obtain the optimized geometries and molecular orbitals of TBT-2(1P-DPA), TBT-2(2P-DPA), and TBT-2(TP-DPA) (FIG. 3b). As for the optimized geometries, TBT-2(1P-DPA) and TBT-2(2P-DPA) with the phenyl rings directly linked to the TBT unit exhibit twisted molecular backbone, where the dihedral angles between the TBT unit and the phenyl rings are 30-35°. Moreover, the two phenyl rings in the r-bridges of TBT-2(2P-DPA) also form a twisting angle of ˜30° due to the steric hindrance. In comparison, the thiophene rings of TBT-2(TP-DPA) generate much smaller dihedral angles of ˜2° and ˜16° with the adjacent TBT unit and phenyl rings, respectively, resulting in a coplanar central part and twisted wings of the molecule. Consequently, the different planarity of these molecules significantly affects the conjugation and D-A interactions. It can be observed from the HOMO/LUMO distributions that TBT-2(1P-DPA) shows typical D-A characters, which can be seen as the reference. The additional phenyl rings of TBT-2(2P-DPA) block the conjugation between the DPA and TBT units, where no LUMO locates at the N atoms at the DPA units. On the contrary, the thiophene rings of TBT-2(TP-DPA) not only extend the conjugation length owing to the fewer twisting angles but also strengthen the intramolecular charge transfer (ICT) due to their more electron richness. As a result, the HOMO-LUMO gaps of the molecules follow the sequence of TBT-2(2P-DPA), TBT-2(1P-DPA), and TBT-2(TP-DPA), more specifically, 1.70, 1.68, and 1.37 eV, respectively. The calculation results indicate that the r-bridges can alter the electronic structures of the molecules, which in turn regulate the optical properties of the materials.

### Example 2—Photophysical, Photodynamic, and Photothermal Properties

To testify the demonstration of molecular simulation results, we then measure the absorption and emission spectrum of TBT-2(1P-DPA), TBT-2(2P-DPA), and TBT-2(TP-DPA) in various polar solvents. It turns out that TBT-2(1P-DPA) and TBT-2(2P-DPA) possess absorption peaks at 580 and 660 nm in THE solution, respectively, and their absorption both ended up to 800 nm (FIG. 3c). However, the absorption peak of TBT-2(TP-DPA) was around 800 nm, and the whole absorption wavelength can be extended to NIR-I region (FIG. 3c), which shows potential as an agent for NIR-II imaging. Expectedly, the emission spectrum of TBT-2(1P-DPA), and TBT-2(2P-DPA) both ends below 1000 nm, while the emission maximum of TBT-2(TP-DPA) was 1046 nm when upon 808 nm excitation (FIG. 3d). Results indicated that with the increase of twisted degree, the absorption and emission spectrum of TBT-2(1P-DPA), TBT-2(2P-DPA), and TBT-2(TP-DPA) presented an increasing trend in wavelengths (FIGS. 3c and 3d), among which TBT-2(TP-DPA) displayed the largest Stokes shift (FIG. 3e). The experimental characterization is in consistency with our theoretical hypothesis, which made TBT-2(TP-DPA) ideal candidates as the template of NIR-II theranostic agents.

Under 808 nm excitation, the absolute quantum yield of TBT-2(TP-DPA) in the NIR region was determined to be 10.4% in toluene. The QY value of the molecule in other solvents and its solid all indicated its superior fluorescence emission capability in the NIR region, making it the firstly qualified probe for in vivo imaging.

### Example 3—Construction of the Nanoaggregates and their Multifunctionality Potential

According to Jablonski diagrams, the acquisition of NIR fluorophores is always a win-win strategy for phototheranostics. With the bathochromic emission quenched in the NIR region, we can make the best use of the nonradiative decay for multimodal theranostics, such as photothermal or photoacoustic imaging and related therapy. In biological applications, polymeric nanoprecipitation is commonly used to bring hydrophobic molecules into biological media. Among them, 1, 2-Distearoyl-sn-glycero-3-phosphoethanolamine-Poly(ethylene glycol) (DSPE-PEG) are widely used in drug delivery. Its biocompatibility, biodegradability and amphiphilicity have made them ideal nanoparticle shells for contrast agents including AlEgens.

Such encapsulation can, in turn, trigger the restriction of intermolecular motion, endowing AlEgens with stronger fluorescence emission when aggregated. Furthermore, thanks to the soft surfactants, the inner parts of the nanoaggregates are tightly packed while the outer part molecules close to the surface of the nanoparticles are loosely packed, which is versatile and feasible for aggregates to achieve radiative and nonradiative decay simultaneously. Inspired by the previous design, we simply encapsulated TBT-2(TP-DPA) with DSPE-PEG2000 (FIG. 4a). The nanoaggregates of TBT-2(TP-DPA) were prepared with an average size of around 32 nm and evenly morphology shown by dynamic laser scanning (DLS) and transmission electron microscopy (TEM) (FIG. 4b). The absorption and emission spectrum of the nanoaggregates were recorded (FIG. 4c), which is similar with that of pure organic molecules.

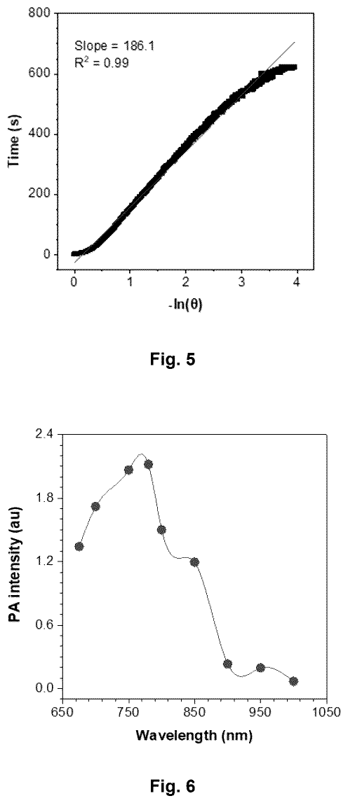

To test the multifunctionality potentials of the nanoaggregates, heat generation capability was first evaluated by calculating their photothermal conversion efficiency (PCE). As expected, the TBT-2(TP-DPA) nanoaggregates performed good photothermal conversion capability, with temperature increasing to above 60° C. when suspended in water at a concentration of 1 mg mL⁻¹ upon 5-min 808 nm continuous laser irradiation (FIG. 4d). The PCE value of the nanoaggregates above were calculated to be 51% (FIG. 5). When applying pulse laser, the photoacoustic response was prompted due to the photoinduced thermoacoustic processes. The photoacoustic spectrum of nanoaggregates in water was measured to be consistency with that of absorption properties, which ranges from 650-1000 nm and has strong photoacoustic signals at 808 nm (FIG. 6). Particularly, the photoacoustic brightness increases gradually with the nanoaggregates concentration increasing (FIG. 4e, inset). The linear relationship between photoacoustic signals and the nanoaggregates concentration can ensure the rationality of quantifying nanoaggregates accumulation via PAI (FIG. 4e). These characteristics can initially satisfy the needs for photothermal imaging, photoacoustic imaging, and photothermal therapy.

### Example 4—Fast Delivery Enabled by Photoinduced Thermoacoustic Processes

It is worth mentioning that in nonradiative decay, the acoustic wave from heat generation can not only assist photoacoustic imaging (PAI), but also be a guiding force for particle delivery in vitro and in vivo. Moreover, it has been validated that many kinds of nanoparticles with the photoacoustic response, no matter ICG, small molecules, polymers or AuNPs, can be triggered by this photoacoustic radiation force. Hence, we evaluated the delivery possibilities and efficiency enabled by photoinduced thermoacoustic (PTA) processes of TBT-2(TP-DPA) nanoaggregates in vivo. Bilateral xenografted 4T1 tumor-bearing mice were employed as the solid tumor models. A dual-wavelength optical-resolution photoacoustic microscopy (ORPAM) system was set up to assist the nanoaggregates delivery towards tumor (808 nm pulse laser), and to simultaneously visualize the structure of blood vessels (532 nm) and nanoaggregates accumulation (808 nm) in or around tumor sites.

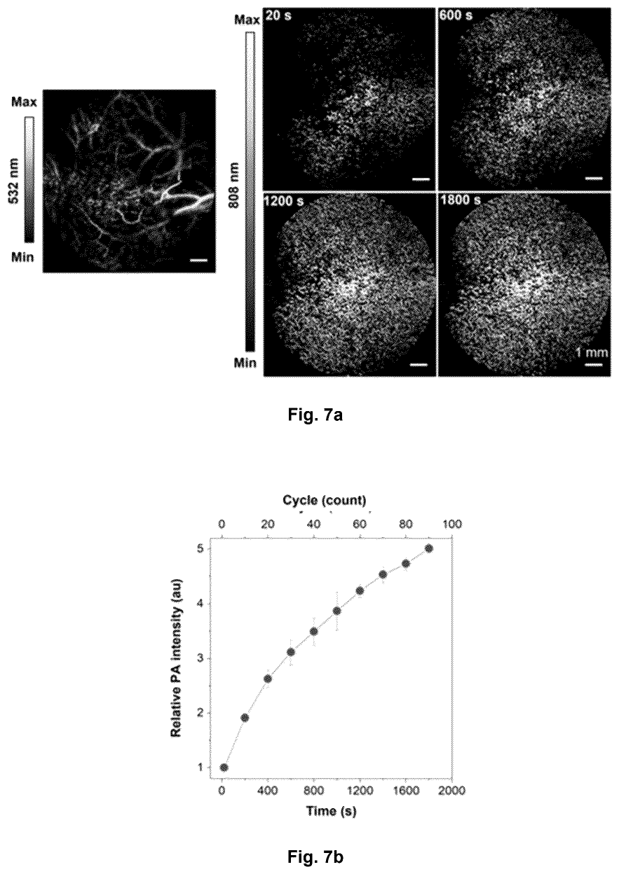

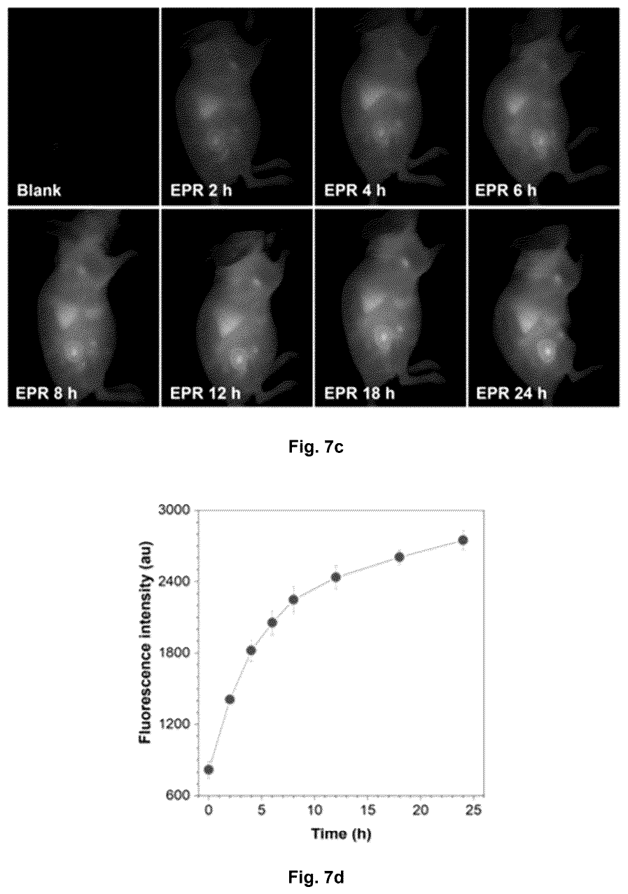

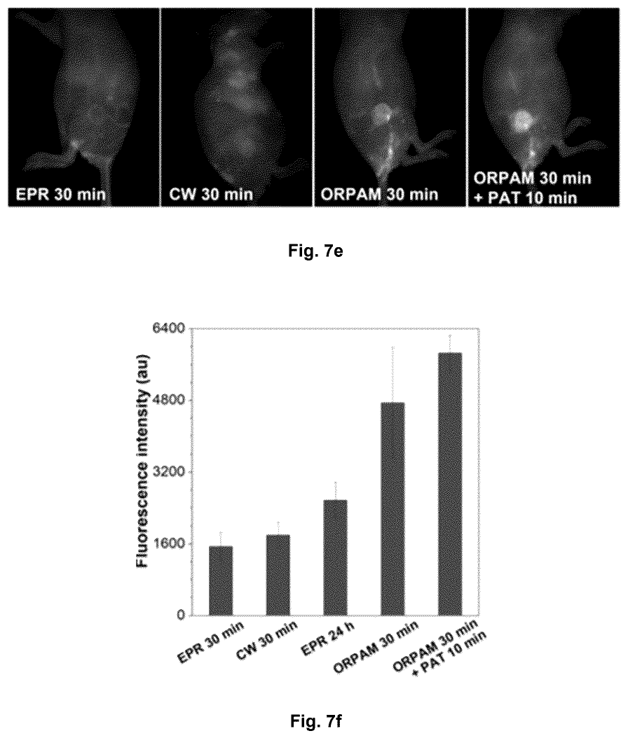

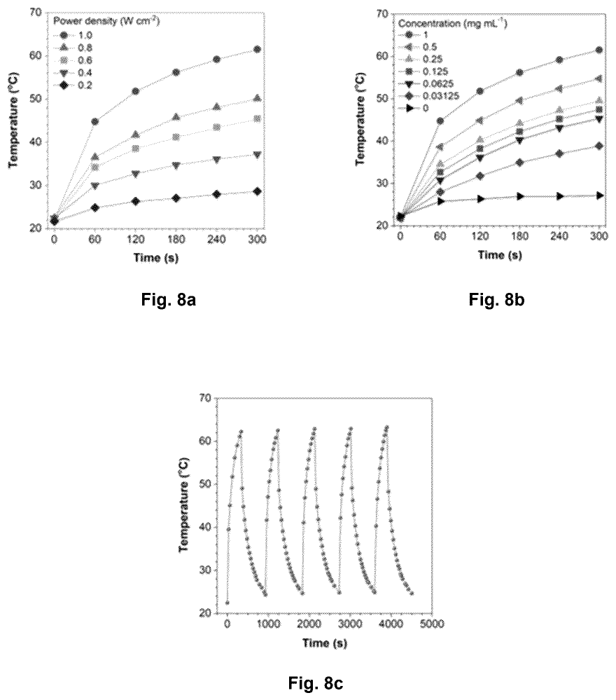

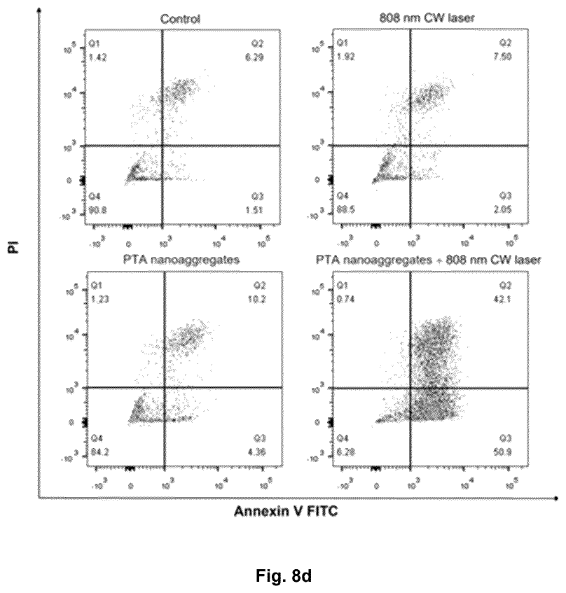

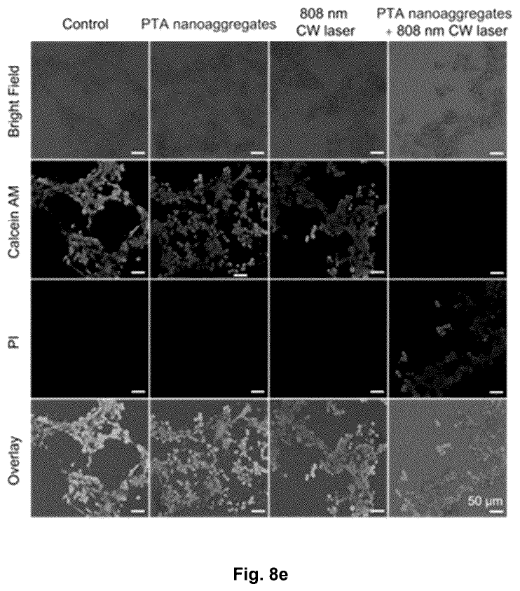

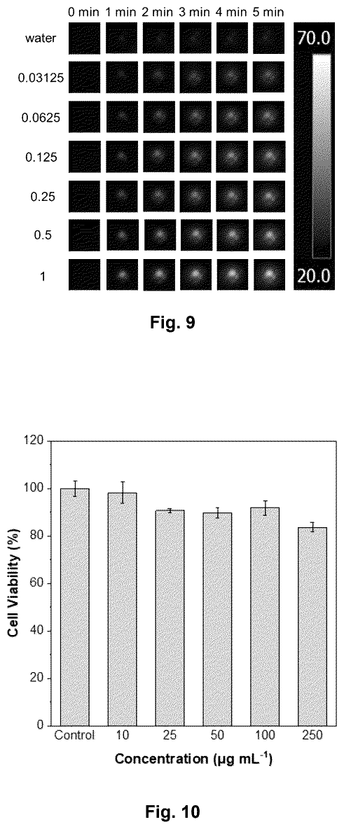

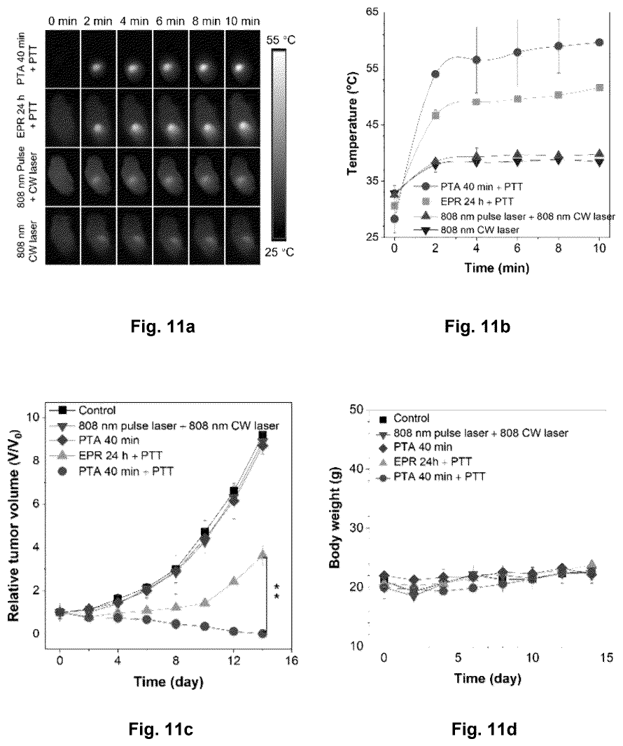

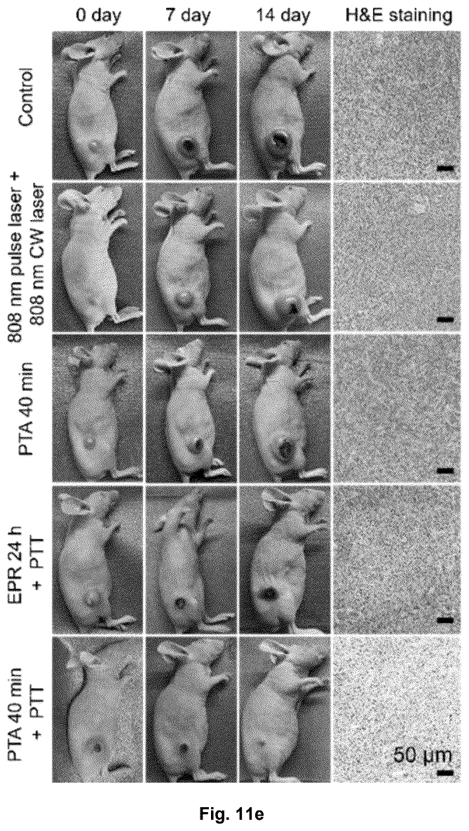

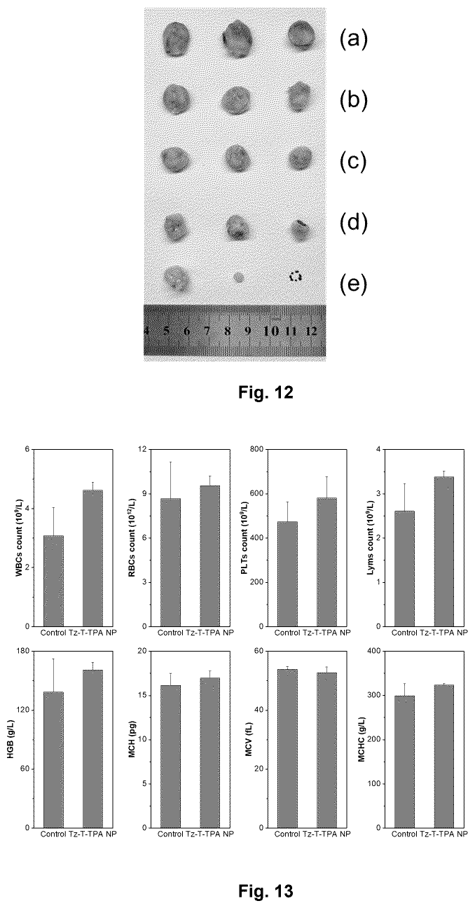

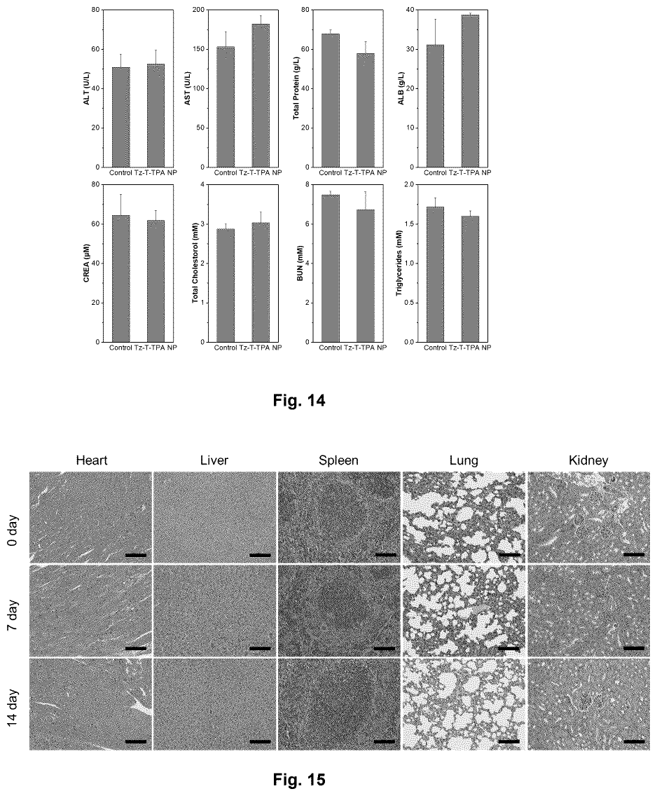

After intravenous injection of the nanoaggregates, we took both tumors on the same mouse as the PTA experimental group and the EPR control group. On the side of the PTA tumor, we established a sequential laser scanning for 30 min (20 s/cycle) using the 808 nm pulse laser to deliver nanoaggregates by PTA processes. As is seen in FIG. 7a, the nanoaggregates accumulated more and more in the solid tumor overtime, and almost distributed spread the area when scanned for 30 min, which is effective. Quantitative results of the time-dependent nanoaggregates accumulation within half an hour by photoacoustic signals can be seen in FIG. 7b.

To study the precision of this PTA-enabled delivery, we also imaged a larger field of view using NIR-II fluorescence emitting from the nanoaggregates. On the EPR side, there was a nearly negligible fluorescent signal in the tumor after the same 30 min interval. However, the fluorescence on the PTA side can clearly distinguish the tumor and surrounding area, and even more signals came out after another 10 min laser scanning by photoacoustic tomography (PAT), which can facilitate delivery in deeper tissues (FIG. 7e). Since the nanoaggregates are only encapsulated with DSPE-PEG without any other targeting modification, the results can validate the feasibility of PTA-driven delivery of the TBT-2(TP-DPA) nanoaggregates, because PTA processes can enhance vascular permeability in the tumor tissue.

Next, to compare the effectiveness of nanoaggregates accumulation in EPR and PTA effect, we then administered the same dose of nanoaggregates via tail vein injection and observed the time-dependent fluorescence signals in another group of mice. Under barely EPR effect, the fluorescence emission in the tumor reached its maximum at 24 hours post-injection (FIGS. 7c and 7d), but it was still much weaker than that of the PTA group within one hour (FIGS. 7e and 7f). Quantitative results demonstrated that the signal intensity in the ORPAM and ORPAM+PAT group was nearly 3-4 times higher than that in the EPR group. The fluorescence signals from the ORPAM+PAT group were still 2-fold as the EPR group when giving them 24 hours to accumulate (FIG. 4k), proving the high efficiency of this PTA-enabled delivery manner. Notably, in this study, we first evaluate the delivery possibility of continuous laser energy towards solid tumors, by irradiating mice tumors for 30 min upon 808 nm CW laser. Fluorescence signals from the CW group indicated that the continuous wave does offer a bit more energy to move the particles forward. However, the outcome is just slightly different from the EPR group and far below the delivery efficiency of the pulse laser. It suggested that the transient enhancement of vascular permeability can be only triggered by photoinduced thermoacoustic processes occupied by the pulse laser, excluding the interference of heat generation.

### Example 5—In Vitro Evaluation of the Photothermal Effect

Based on the tuning of the TICT effect on molecular design, it is easier for NIR fluorophores to generate heat. Since TICT can weaken the fluorescence intensity but simultaneously elevate their photothermal effect, balancing the radiative and nonradiative decay of one single molecule makes it a multifunctional theranostic agent. The photothermal effect of the TBT-2(TP-DPA) nanoaggregates depends on power density (FIG. 8a) and concentration (FIG. 8b and FIG. 9), which provided the basis for optimization of the next in vitro experiments. It is noteworthy that even when experienced continuously cyclic 5 heating/cooling processes, the nanoaggregates still exhibited excellent photostability (FIG. 8c).

To act as a qualified theranostic agent, the materials should possess no cytotoxicity when the laser is off. Therefore, we evaluate the dark toxicity of the nanoaggregates on 4T1 cells with CCK-8 assay. It shows that the viabilities of 4T1 cells can keep over 80% even at concentration of 250 μg mL⁻¹ when incubated with TBT-2(TP-DPA) nanoaggregates for 24 h, presenting good biocompatibility (FIG. 10). However, when subsequently irradiated with an 808 nm continuous laser for 10 min, the cells incubated with the nanoaggregates showed apoptosis. Flow cytometry results indicated that about 42.1% of the cells would experience late apoptosis and 50.9% were early apoptotic after the as-described treatment (FIG. 8d). Meanwhile, the apoptotic cell percentage of the control group, the 808 nm laser irradiation group and the barely nanoaggregates without irradiation group were all below 15%. The significant cell-killing effect via photothermal treatment can guarantee effective cancer cell ablation for later in vivo experiments.

We also visualized the cell morphology under the PTT treatment using Calcein AM and propidium iodide (PI) as live/dead cell assay. Confocal imaging revealed that the TBT-2(TP-DPA) nanoaggregates+laser group emitted strong red fluorescence for dead cells, while the other groups including the control only emitted green fluorescence representing live cells (FIG. 8e), suggesting the consistency and reliability of our in vitro PTT results.

### Example 6—In Vivo Evaluation of PTA-Enhanced Photothermal Therapy

After the confirmation of nanoaggregates accumulation enabled by photoinduced thermoacoustic processes and the aggregates' photothermal effect on cancer cells, we then have a solid reason to investigate their therapeutic effect in vivo. Several 4T1 tumor-bearing mice were randomly divided into 5 groups. 1) TBT-2(TP-DPA) nanoaggregates+808 nm pulse laser+808 nm CW laser (PTA+PTT); 2) TBT-2(TP-DPA) nanoaggregates+808 nm CW laser (EPR 24 h+PTT); 3) TBT-2(TP-DPA) nanoaggregates+808 nm pulse laser (PTA); 4) 808 nm pulse laser+808 nm CW laser; and 5) PBS (control).

To visualize the actual photothermal effect in vivo in real-time, IR thermal camera was set up to monitor the IR imaging on all the mice in the group 1), 2), 4) and 5) (FIG. 11a). As is quantified in FIG. 11b, the tumor temperature in the PTA+PTT group were elevated to 53° C. after only 2 minutes of 808 nm laser irradiation, and further rise to nearly 60° C. in the following 8 min. In comparison, despite we leave 24 hours for the EPR group to accumulate sufficient nanoaggregates toward the tumor, they only climbed to 51° C. within 10 min under the same laser treatment (FIG. 11b). On the contrary, no matter treated with barely 808 nm CW laser (group 5, control) or plus 808 nm pulse laser (group 4), if there were no nanoaggregates injection, the laser themselves would cause slightly negligible temperature change on the tumor from the initial mice body temperature, and the final temperature below 40° C. would not influence the morphology of tumor tissue (FIG. 11b).

During the following 14 days, the digital images, tumor volumes, and body weights of mice in all 5 groups were recorded and analysed. As is displayed in FIG. 11c, 11e and FIG. 12, the solid tumor on mice in the PTA+PTT group were gradually shrunk and obviously eliminated without any reoccurrence. Nevertheless, there is almost no therapeutic effect in the groups with only 808 m CW or CW plus pulse laser irradiation but without nanoaggregates administered (group 4 as control and group 5). In comparison, despite that the EPR+PTT group exhibited an inhibition effect in the first 4 days, the tumor would still regrow afterward, which can be ascribed to the incomplete killing of cancer cells in barely EPR.

The body weight of mice in all groups kept stable within a normal range during the observation (FIG. 11d). In addition, more severe signs of necrosis in the PTA+PTT group can be seen according to the haematoxylin and eosin (H&E) staining (FIG. 11e). Moreover, the therapeutic process of PTA+PTT only took 40 min of 808 nm pulse laser scanning, while the traditional EPR for even 24 hours before PTT cannot reach the same level of tumor-killing effectiveness as PTA-guided delivery. The biosafety of our designed TBT-2(TP-DPA) nanoaggregates administered in the mice were also investigated and analysed via blood tests and H&E staining (FIG. 13-15). All the blood routine and serum biochemistry results show that there was no significant difference between the nanoaggregates administered group at 14 days post injection and the control group. The main organs collected from the mice sacrificed on day 14 post-injection showed no obvious lesion or damage, including the heart, liver, spleen, lung, and kidney.

These results collectively confirmed the biosafety of TBT-2(TP-DPA) nanoaggregates for potential bio-applications. In summary, this work offers new NIR-absorbing nanoaggregates based on small molecules, which not only perform promising multifunctional phototheranostics (FLI, PAI, PTI, and PTT) effectiveness but are also adapted to the approach and platform of drug delivery enabled by photoinduced thermoacoustic processes. With this strategy, there is a reason to believe that other NIR-absorbing small molecule-based nanoaggregates are also promising in this PTA-guided fast delivery and efficient cancer treatment, which would make up the shortcoming of EPR and save some time for tumor-targeting design.

The above embodiments are only used to illustrate the principles of the present invention, and they should not be construed as to limit the present invention in any way. The above embodiments can be modified by those with ordinary skill in the art without departing from the scope of the present invention as defined in the following appended claims.

What is claimed is:

1. A fluorescent compound exhibiting aggregation-induced emission properties, the fluorescent compound having a backbone structural formula selected from the group consisting of: 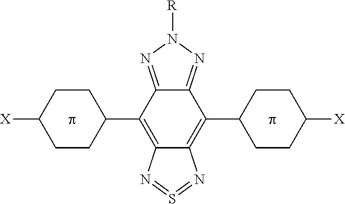
wherein R is selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C(O)—O—, or —C≡C—and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each π is independently selected from the group consisting of: 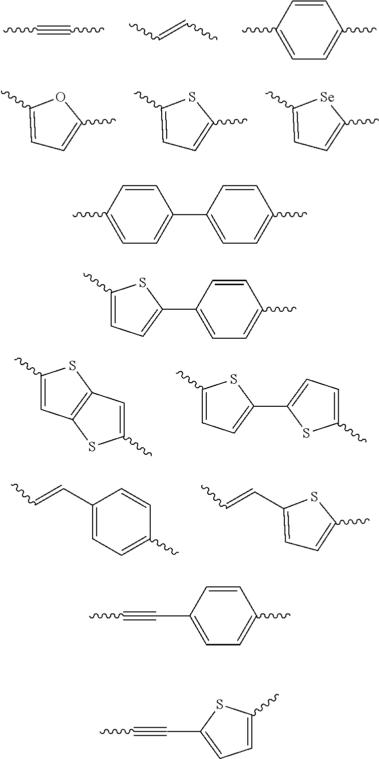
wherein each X is independently selected from the group consisting of:

[chemical structure]

2. The fluorescent compound of claim 1, wherein the fluorescent compound exhibits near-infrared (NIR) emission.

3. The fluorescent compound of claim 1, wherein the fluorescent compound comprises:

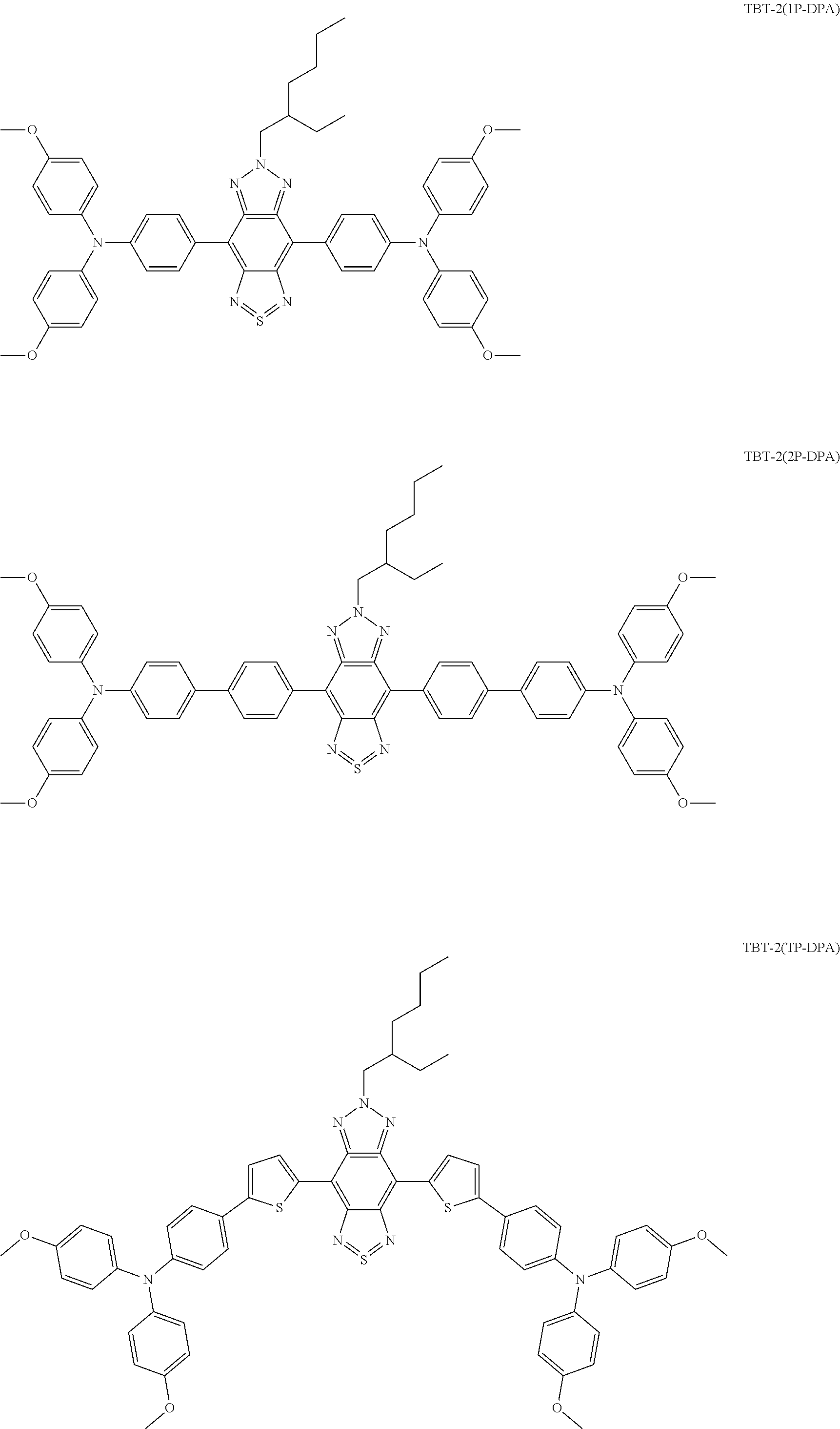

4. The fluorescent compound of claim 1, wherein the fluorescent compound is in nanoparticle form.

5. A theranostic nanoaggregates comprising:
nanoparticle comprising the fluorescent compound of claim 1; and
polymer nanoshell, wherein the polymer nanoshell covers the nanoparticle, the polymer nanoshell is formed by using a polymer matrix to encapsulate the nanoparticle.

6. A method of killing cancer cells, comprising:
contacting a target cancer cell with the theranostic nanoaggregates according to claim 5;
imaging the target cancer cell while the theranostic nanoaggregates contacts the target cancer cell using an imaging method selected from the group consisting of fluorescence microscopy, bioluminescence imaging, confocal laser scanning microscopy and photoacoustic microscopy; and
subjecting the target cancer cell to light irradiation while the theranostic nanoaggregates is contacting the target cancer cell converts light to heat to kill the target cancer cell.

7. The method of claim 6, wherein the light irradiation is near-infrared light irradiation.

8. The method of claim 6, wherein the light irradiation is laser irradiation.

9. A method of stopping tumor growth or inhibiting tumor growth or eliminating tumor in a mammal, comprising:
administering the theranostic nanoaggregates of claim 5 to the mammal;
contacting a tumor site with the theranostic nanoaggregates;
locating the tumor site using an imaging method after the tumor site is contacted with the theranostic nanoaggregates;
subjecting the tumor site to an initial pulsed light irradiation to enhance the vascular permeability near the tumor site, so as to drive enough theranostic nanoaggregates to accumulate on the tumor site; and
subjecting the tumor site to a subsequent continuous light irradiation while enough theranostic nanoaggregates at the tumor site converts light to heat to rise the temperature of the tumor site to a desired temperature, so as to stop or inhibit the growth of the tumor, or to eliminate the tumor.

10. The method of claim 9, wherein the theranostic agent is administered by intravenous injection.

11. The method of claim 9, wherein the imaging method comprises at least one of fluorescence microscopy, bioluminescence imaging, confocal laser scanning microscopy, and photoacoustic microscopy.

12. The method of claim 9, wherein the light irradiation is near-infrared light irradiation.

13. The method of claim 9, wherein the light irradiation is laser irradiation.

14. The method of claim 9, wherein the desired temperature is higher than 55 degrees Celsius.

15. The method of claim 9, wherein the combined duration of the initial pulsed light irradiation and the subsequent continuous light irradiation is less than one hour.

16. The method of claim 9, wherein the initial pulsed light irradiation comprises a sequence of light pulses more than 3.

17. The method of claim 16, wherein the peak irradiation intensity is higher than 0.5 W/cm² and lower than 100 W/cm².

18. The method of claim 16, wherein the effective pulse length is between 5 microseconds to 5 seconds.

19. The method of claim 16, wherein the off time between each two adjacent light pulses sufficient to allow the transient heat of the vasculature near the tumor site to be dissipated into the surrounding media, so as to avoid overheating and damaging the vasculature near the tumor site.

20. A method of stopping tumor growth or inhibiting tumor growth or eliminating tumor in a mammal, comprising:
administering theranostic agents to the mammal;
contacting a tumor site with the theranostic agents;
locating the tumor site using an imaging method after the tumor site is contacted with the theranostic agents;
subjecting the tumor site to an initial pulsed light irradiation to enhance the vascular permeability near the tumor site, so as to drive enough theranostic agents to accumulate on the tumor site; and
subjecting the tumor site to a subsequent continuous light irradiation while enough theranostic agents at the tumor site converts light to heat to rise the temperature of the tumor site to a desired temperature, so as to stop or inhibit the growth of the tumor, or to eliminate the tumor.

21. The method of claim 20, wherein the theranostic agents is administered by intravenous injection.

22. The method of claim 20, wherein the theranostic agents comprises nanoparticles or nanoaggregates with photoacoustic response.

23. The method of claim 20, wherein the theranostic agents comprises ICG, small molecules, polymers, AuNPs or AlEgens.

24. The method of claim 20, wherein the imaging method comprises at least one of fluorescence microscopy, bioluminescence imaging, confocal laser scanning microscopy, and photoacoustic microscopy.

25. The method of claim 20, wherein the light irradiation is near-infrared light irradiation.

26. The method of claim 20, wherein the light irradiation is laser irradiation.

27. The method of claim 20, wherein the desired temperature is higher than 55 degrees Celsius.

28. The method of claim 20, wherein the combined duration of the initial pulsed light irradiation and the subsequent continuous light irradiation is less than one hour.

29. The method of claim 20, wherein the initial pulsed light irradiation comprises a sequence of light pulses more than 3.

30. The method of claim 29, wherein the peak irradiation intensity is higher than 0.5 W/cm² and lower than 100 W/cm².

31. The method of claim 29, wherein the effective pulse length is between 5 microseconds to 5 seconds.

32. The method of claim 29, wherein the off time between each two adjacent light pulses sufficient to allow the transient heat of the vasculature near the tumor site to be dissipated into the surrounding media, so as to avoid overheating and damaging the vasculature near the tumor site.
